(19) United States
(12) Patent Application Publication
(10) Pub. No.: US 2025/0073460 A1
(43) Pub. Date: Mar. 6, 2025

(54) INTERFERENTIAL VESTIBULAR STIMULATION TO MINIMIZE MOTION SICKNESS

(71) Applicant: Adeia Guides Inc., San Jose, CA (US)

(72) Inventor: Aldis Sipolins, Somerville, MA (US)

(21) Appl. No.: 18/240,962

(22) Filed: Aug. 31, 2023

(51) Int. Cl. A61N 1/32; A61B 5/11; A61N 1/36

(52) U.S. Cl. CPC A61N 1/323; A61B 5/11; A61N 1/36036

(57) ABSTRACT

The present disclosure is directed to systems and methods for using interferential stimulation to reduce or prevent symptoms of motion sickness in a closed system. In an embodiment the systems and methods receive an acceleration data stream, calculate parameters of interferential stimulation likely to cause the sensation of acceleration within a person's vestibular system that matches the acceleration the person's eyes perceive. The systems and methods deliver the interferential stimulation and measure a resulting head movement. The systems and method then compare that movement to a predicted movement. If the movement is not as predicted, the systems and methods update the parameters and deliver a second interferential stimulation until a desired affect is reached.

### BACKGROUND OF THE INVENTION

The present system is related to using closed-loop interferential stimulation techniques to impact perceived acceleration.

### SUMMARY OF THE INVENTION

The present disclosure relates to producing a closed-loop system of interferential stimulation to counteract stimuli causing motion sickness. The closed-loop system may be incorporated into a headband or headset which senses acceleration and delivers interferential stimulation to reach the vestibular system of a wearer.

Existing treatments for motion sickness offer a number of options including over the counter medication, wristbands, and home remedies. These treatments do not target known causes of motion sickness, however. As a result, the efficacy of these treatments remains low. Further over the counter medication can cause drowsiness and impair both motor and cognitive function. These medications also may run out quickly and must be continually purchased. There remains a need for an effective motion sickness treatment that targets the causes of motion sickness, does not impair function, and is readily available.

Motion sickness is often the result of the brain receiving conflicting information regarding motion. For example, a passenger who is reading in a moving vehicle (or looking at any other object in a vehicle) will sense acceleration through his or her vestibular system, or the inner ear, as the vehicle accelerates. However, that same passenger's vision will indicate that the passenger is not moving because the passenger is looking at a page that appears still (because it's accelerating in unison with the passenger). The conflicting information causes the individual to experience motion sickness.

The present disclosure describes systems and techniques for alleviating symptoms of motion sickness by targeting these causes, herein referred to as an Interferential Vestibular Stimulation (IVS system). In some embodiments, the IVS system delivers interferential stimulation that acts on the individual's vestibular system and causes them to perceive acceleration that aligns with visual motion cues. The interferential stimulation may be alternating current which is more precise than direct current. The alignment between perceived acceleration caused by stimulation and visual motion cues then reduces or prevents symptoms of motion sickness. Moreover, the perceived acceleration causes the individual to move his or her body or head in response, and the direction of movement can be used to indicate the direction of perceived acceleration caused by stimulation. In some approaches, the magnitude and direction of the movement is captured by the IVS system (e.g., using an accelerometer sensor) and is used to continuously adjust the interferential stimulation.

For example, the present IVS system reaches a passenger's or other individual's vestibular system using interferential stimulation delivered through electrodes placed in contact with the head (e.g., around both ears) of the passenger. In some instances, the interferential stimulation neutralizes the sensation of acceleration. In one example, if a passenger in a car is reading when the car slows in response to traffic, the passenger's vestibular system will detect, and the passenger will perceive forward acceleration. The present disclosure may then deliver interferential stimulation causing the passenger to perceive no acceleration at all. When traffic has passed and the vehicle accelerates to full speed, the present disclosure may similarly apply interferential stimulation causing the passenger to perceive no acceleration. The result is a smoother ride for the passenger with no disruptions.

In another embodiment, the interferential stimulation causes the user to perceive acceleration that matches visual cues. For example, in a situation where an individual uses virtual reality to experience a roller coaster, the vestibular system and vision of the individual receive conflicting information about acceleration. Information the individual's brain receives from the eyes tell the individual that there is acceleration while the vestibular system detects none. This causes motion sickness. The present disclosure in this embodiment delivers interferential stimulation causing the individual to perceive acceleration through the vestibular system that matches the visual experience. The acceleration for the interferential stimulation may be based on metadata or a data feed from the VR content. The result is an enhanced immersive experience with no motion sickness side effects.

### BRIEF DESCRIPTION OF THE DRAWINGS

FIG. 1A shows an example process in accordance with an embodiment of the present disclosure;

FIG. 1B shows an example process in accordance with an embodiment of the present disclosure;

FIG. 2A shows an example anatomy of the vestibular system;

FIG. 2B shows example directions of acceleration in accordance with an embodiment of the present disclosure;

FIG. 2C shows another view of example directions of acceleration in accordance with an embodiment of the present disclosure;

FIG. 3 shows an illustration of interferential stimulation in accordance with an embodiment of the present disclosure;

FIG. 4A shows example areas of brain stimulation in accordance with an embodiment of the present disclosure;

FIG. 4B shows electrode configurations in accordance with an embodiment of the present disclosure;

FIG. 5 shows an example architecture of an embodiment of the present disclosure;

FIG. 6 shows an example headset in accordance with an embodiment of the present disclosure;

FIG. 7A shows flowchart of an example process in accordance with an embodiment of the present disclosure;

FIG. 7B shows flowchart of an example process in accordance with an embodiment of the present disclosure; and

FIG. 8 shows flowchart of a second example process in accordance with an embodiment of the present disclosure.

### DETAILED DESCRIPTION

The present system is directed to management of symptoms of motion sickness and more particularly to using interferential stimulation techniques to counteract perceived acceleration. Generally speaking, existing remedies for motion sickness do not target the mechanisms that cause the illness and may impair cognitive and motor functions. The systems and methods of the present disclosure address the causes of motion sickness to give effective, widely applicable relief and prevention without requiring medication and without unwanted side effects.

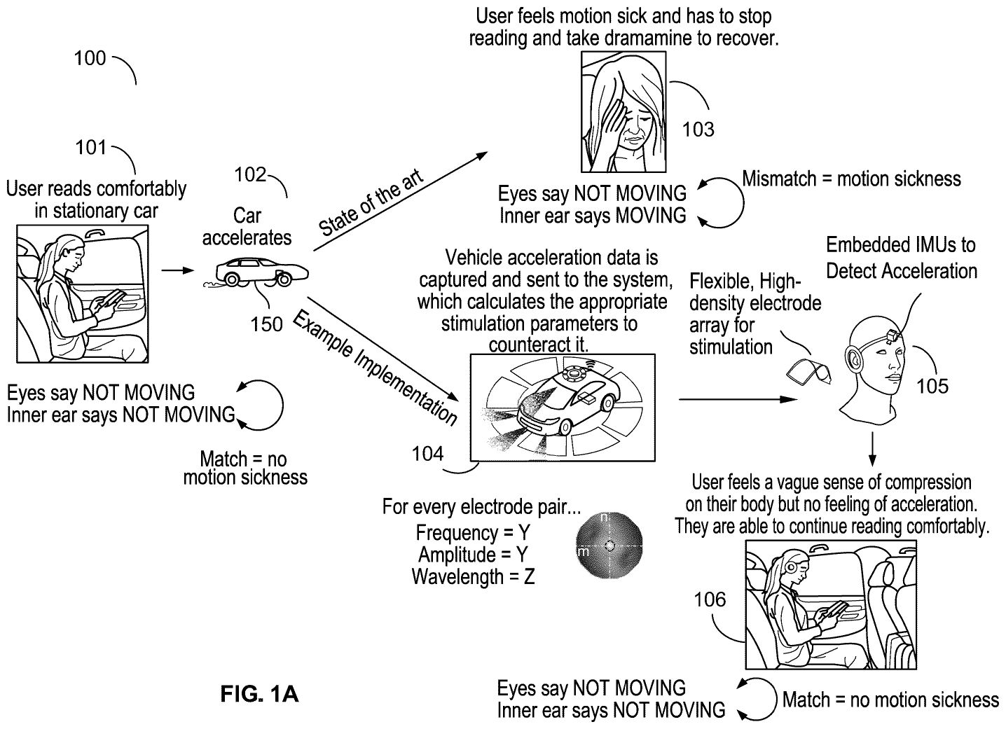

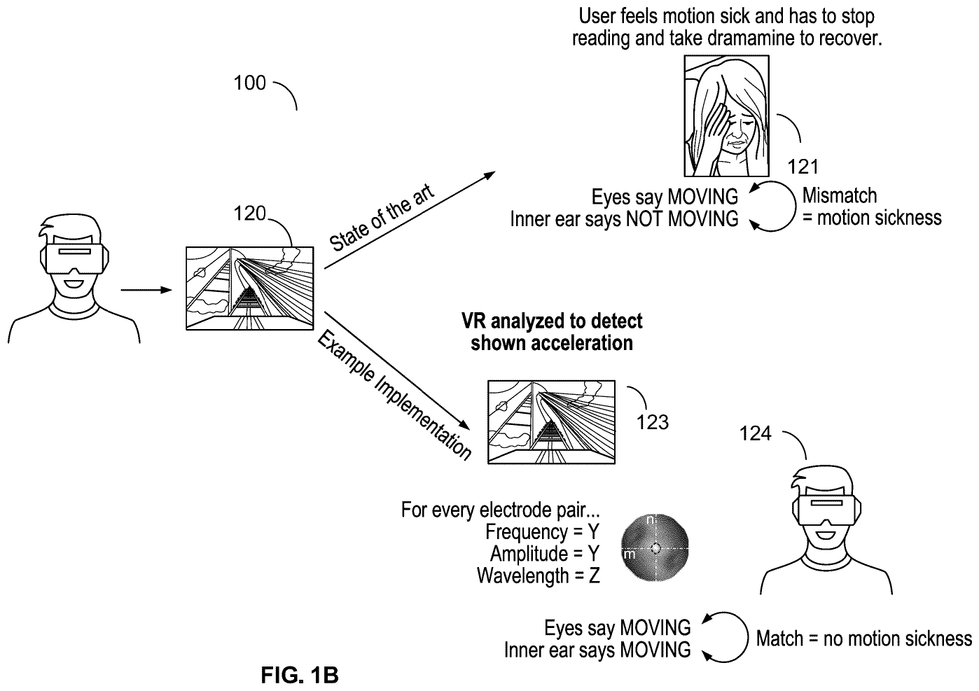

An example IVS system 100 and process of the proposed systems and methods are described in FIG. 1A. FIG. 1A shows an embodiment in a vehicle but IVS system 100 may apply in any scenario involving a conflict of acceleration information the eyes perceive and the vestibular system perceives. In a still automobile 150, or other vehicle such as a train, boat, or airplane, passengers are able to watch videos, read a map, write directions, or participate in other activities without incidence. Such a situation is shown at step 101 where automobile 150 does not move. Passengers are able to read, or participate in other activities, without feeling motion sick. At step 102 the automobile 150 begins to accelerate. When the automobile 150 speeds up or slows down, a passenger in some embodiments detects this acceleration both visually and through the passenger's inner ear, or vestibular system, which translates interpretation of movements by way of acceleration to maintain a passenger's balance.

When the automobile 150 accelerates, the vestibular system of a passenger who is reading senses the motion. However, the field of view of this reading passenger tells the passenger that he or she is still. This discrepancy between the senses of the passenger's vestibular system sense and the senses of the passenger's ocular system causes the passenger to feel sick as illustrated in step 103. The passenger may then either stop reading or try to improve their symptoms.

The present disclosure offers a solution to motion sickness experienced due to a mismatch between senses of the vestibular system and senses of the ocular system by way of alternative using interferential stimulation delivered to the vestibular system through electrical pulses in a closed-loop system. This stimulation in some embodiments overrides the vestibular system of a person, for example specifically in the example of FIG. 1A a passenger, to cause it to detect a prescribed acceleration, rather than actual acceleration of the body in physical space. The sense of acceleration in some embodiments causes a person to move his or her head in response to the vestibular system sensing acceleration. By creating vestibular stimulation that aligns with acceleration detected by the eyes, the IVS system 100 of the present disclosure is able to reduce or prevent motion sickness. In an embodiment the IVS system 100 is a closed-loop system that is able to improve its function and accuracy of the vestibular stimulation over time, resulting in a more effective treatment.

At step 104, as automobile 150 accelerates, the IVS system 100 of the present disclosure detects acceleration using IMU(s) (inertial measurement unit or units) integrated into IVS system 100. The IMU may provide to the IVS system 100 an acceleration data stream, which is a continuous or on going flow of acceleration data. IVS system 100 then calculates the appropriate stimulation parameters, that is the parameters that define the electrical currents and fields of the delivered interferential stimulation, that are likely to cause the passenger's vestibular system to perceive that the passenger is still. In some embodiments the calculated parameters are based on a provided model that predicts an appropriate location of the vestibular system and appropriate intensities for stimulating it so. In some embodiments, the IVS system 100 calibrates vestibular stimulation, as discussed below, to determine variables such as vestibular system location prior to determining the parameters. Such parameters in some embodiments include the wavelength, amplitude, and frequency of a current that an electrode delivers to a person's head and vestibular system, as discussed in more detail below. In an embodiment, acceleration detection and interferential stimulation occur via a wearable device or headset, seen at step 105.

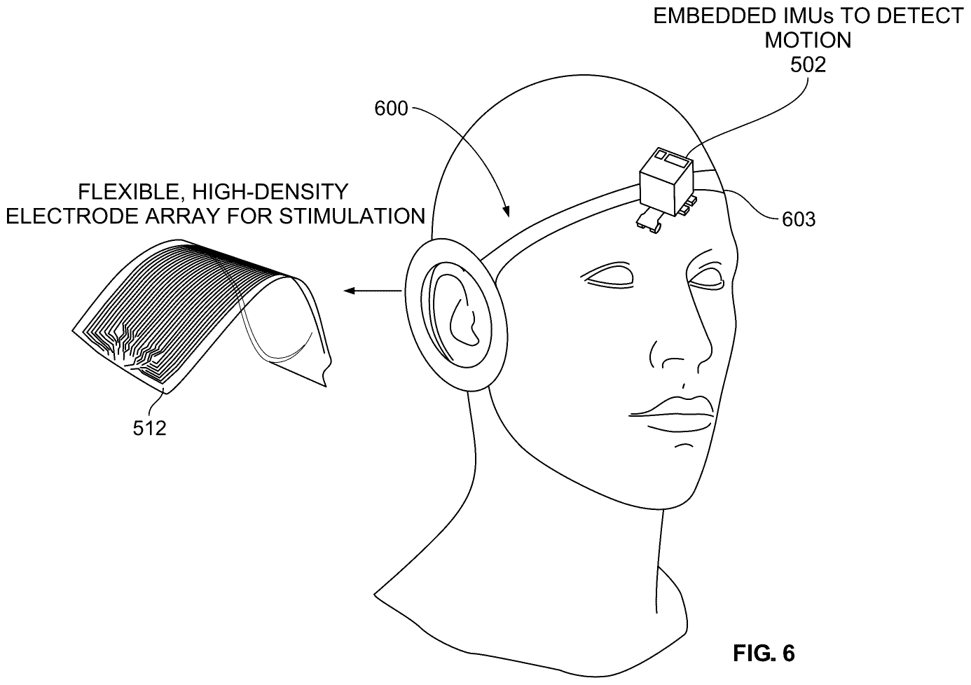

Internal hardware and software on a wearable device, discussed in detail in FIG. 6, collect data regarding the acceleration of the passenger and communicate this information to the larger IVS system 100. In an embodiment IMUs are embedded in the headset and detect acceleration of the passenger, the vehicle, or both. The IVS system 100 in an embodiment then analyzes the data, and outputs appropriate stimulation parameters describing two or more electrical currents reaching the passenger's head that create an electrical field, the interferential stimulation. Using these parameters, in an embodiment the IVS system 100 delivers, via electrode pairs attached to the head, an interferential stimulation that reaches the vestibular system and is known to cause a sensation of acceleration in line with what the passenger sees. At step 106 the passenger receives the interferential stimulation known to cause a sensation of the appropriate acceleration. The passenger does not sense the acceleration of the vehicle 150 and is able to read, or participate in other activities, comfortably without feeling ill. In an embodiment the IVS system 100 delivers a series of currents with varying intensity and parameters, or modifies the first set of parameters, until the IVS system 100 determines, by way of monitoring a reactionary movement of a person's head, an interferential field having the desired impact, for example the desired perception of acceleration. Such a process may be conducted as a closed-loop, continuously receiving input and adjusting output accordingly until the output matches the intended result. In an embodiment, the IVS system 100 detects the response to the interferential field by measuring acceleration of the passenger or other individual through IMUs connected to the passenger or other individual using IVS system 100. In an embodiment the intensities of the currents are directly related to a confidence level that the current will produce the desired effect. In an embodiment the windows of automobile 150 are tinted to limit conflicting visual stimulation.

In an embodiment, the IVS system 100 includes a calibration phase which occurs before vehicle 150 begins accelerating. The calibration phase determines the expected acceleration a vestibular system perceives for different interferential stimulation parameters. In such an embodiment, the process discussed in FIG. 1 may not include a closed-loop cycle but rather may deliver the interferential stimulation likely to result in the desired perceived acceleration without follow up. Another embodiment may further include at least one IMU in the vehicle 150 which detects the vehicle 150's acceleration and the IVS system 100 may account for the vehicle acceleration in determining the interferential stimulation parameters. Another embodiment does not include at least one IMU in the vehicle 150 and instead the parameters are set according to user settings, preferences, other input, or predetermined information.

FIG. 1B shows an example embodiment of the present disclosure in which the IVS system 100 reduces or prevents motion sickness symptoms in the context of extended reality (e.g., virtual reality). At step 120 an individual wearing an extended reality (XR) headset (e.g., a virtual reality or VR headset) watches, for example, a VR segment portraying the experience of riding a rollercoaster, although the system may benefit a user in any scenario in which visual cues produce motion sickness, including, for example, an Imax segment, a video game, or an augmented reality (AR) display. References herein to an “XR device” or “XR headset” refer to a device providing virtual reality (VR), mixed or merged reality (MR), or augmented reality (AR) functionality (e.g., wherein virtual objects or graphic overlays are provided in addition to real-world objects or environments visible via the device). An XR device may take the form of glasses or a headset. In one approach, shown at step 121, an individual wearing a XR headset will perceive acceleration through visual cues interpreting motion displayed on a screen of the headset, but will not perceive acceleration through the vestibular system. The conflicting information causes the individual to feel motion sick. The present disclosure offers an alternative to the approach shown in step 121. In an embodiment, the disclosed IVS system 100 uses interferential stimulation to create a sensation of acceleration in the vestibular system of the individual that matches the acceleration detected through vision (e.g., a forward/backward acceleration, a lateral acceleration, an up/down acceleration, or an acceleration of 0). At step 123, the IVS system 100 receives acceleration information directly from the roller coaster stream, for example through its metadata. The IVS system 100 then, using this information, determines parameters likely to create feelings of the same acceleration in a vestibular system. The IVS system 100 delivers interferential stimulation as electrical current according to the determined parameters at step 124. At this step the IVS system also refines the interferential stimulation until it determines a match. When the interferential stimulation causes a sensation of vestibular acceleration matching the sense of acceleration seen in the XR feed, the individual no longer feels motion sick.

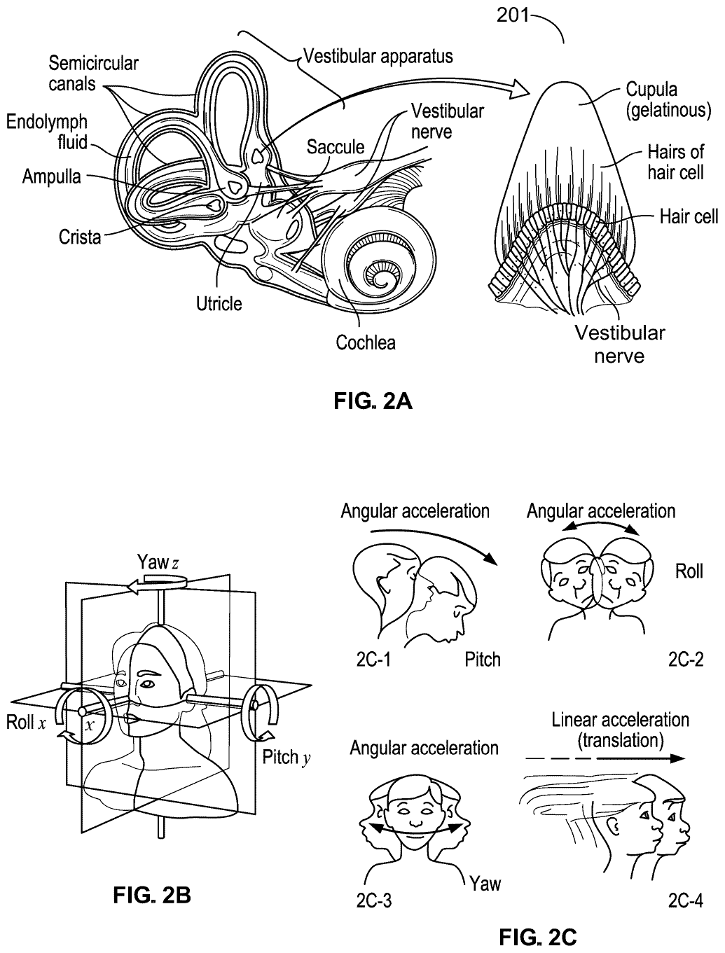

Interferential stimulation impacts a passenger's perception of acceleration by sending signals to the passenger's inner ear or vestibular system 201 seen in FIG. 2A. The vestibular system 201 is a complex collection of mechanical sensors that detect both angular and linear acceleration shown in FIG. 2B. As seen in FIG. 2B, angular or rotational acceleration may be understood as acceleration of any of three angles of orientation: (i) roll (rotation around a longitudinal or x axis), (ii) pitch (rotation around a lateral or y axis), and (iii) yaw (rotation around a vertical or z axis). FIG. 2C illustrates angular accelerations (2C-1-2C3) and linear acceleration (2C-4) from the perspective of two dimensions. Example 2C-1 demonstrates pitch acceleration. Example 2C-2 demonstrates roll acceleration. Example 2C-3 demonstrates yaw acceleration. Example 2C-4 demonstrates linear acceleration (which may occur forward/backward along a longitudinal axis, side-to-side along a lateral axis, or up/down along a vertical axis).

Passengers also receive visual cues about acceleration. Seeing surroundings move indicates to the passenger that he or she is accelerating. However in some scenarios visual perception of acceleration does not align with a passenger's vestibular perception of acceleration. For example, one system of the passenger's body (visual or vestibular) may detect acceleration (e.g., linear or angular) while the other does not. For example, when reading in automobile 150, a passenger will not see motion. The passenger's eyes will be focused on the page in front of the passenger and the passenger will not see, for example, the view of a nearby window in which the surroundings of the passenger are moving. The passenger will only see a still page. However, in this scenario, the vestibular system of the passenger will detect acceleration because the passenger is physically accelerating inside of a moving vehicle that changes speed continuously. The mismatch of information, information from the eyes that the passenger is not accelerating and information from the vestibular system that the passenger is accelerating, will cause the passenger to feel motion sick. Similarly, in an immersive video experience such as when watching a video in an IMAX theatre, an individual's eyes might detect acceleration from the video images, but the individual's vestibular system will not detect any acceleration. As a result of the mismatch of information this individual may also feel motion sick.

The IVS system 100 acts on a passenger's—or other individual's—vestibular system through interferential stimulation. Interferential stimulation is an electrical stimulation technique. It involves administering electrical currents of a desired frequency (e.g., high frequency) by two or more electrodes attached to a head. When the currents meet, they create an electrical field pattern impacting the brain. Adjusting the currents alters where the pattern of the electrical field impacts the head and as a result, careful refinement allows the pattern to target a specific area of the head, including specific regions of the brain and other parts. The currents in some embodiments are too high to negatively affect human tissue. In other words, interferential stimulation enables selective stimulation of specific regions without affecting surrounding tissue. The currents may differ very slightly in the current frequency, to create interference. For example, if one electrode applies 2 kHz current and another applies 2.01 kHz current, they will create an interference pattern with a frequency of 0.01 kHz where those currents meet. When properly calibrated, the resulting interference pattern will have a frequency that can stimulate brain tissue. When properly targeted, the resulting interference pattern can reach areas at a specific distance away from the electrodes. Therefore, the interference can target specific parts of the brain when properly calculated. This is especially true when using multiple electrodes with multiple wavelengths.

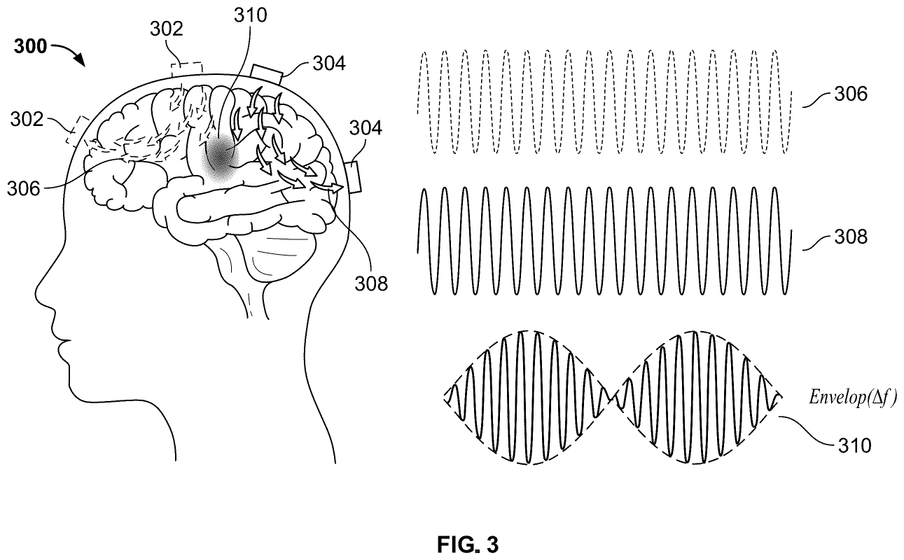

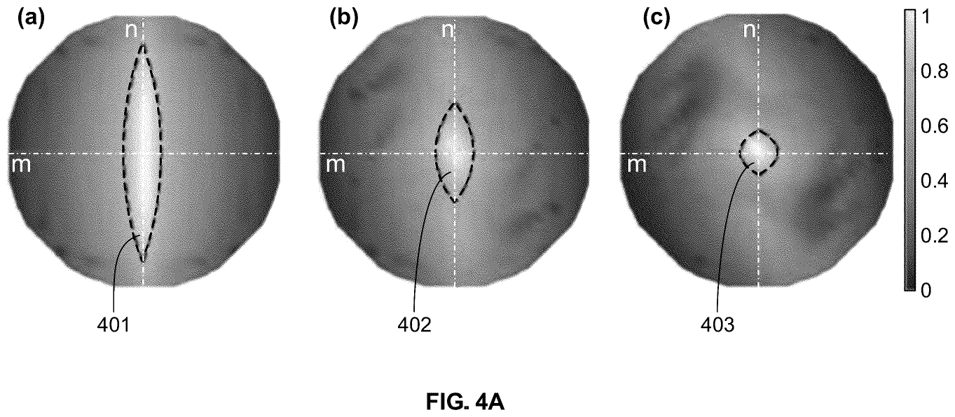

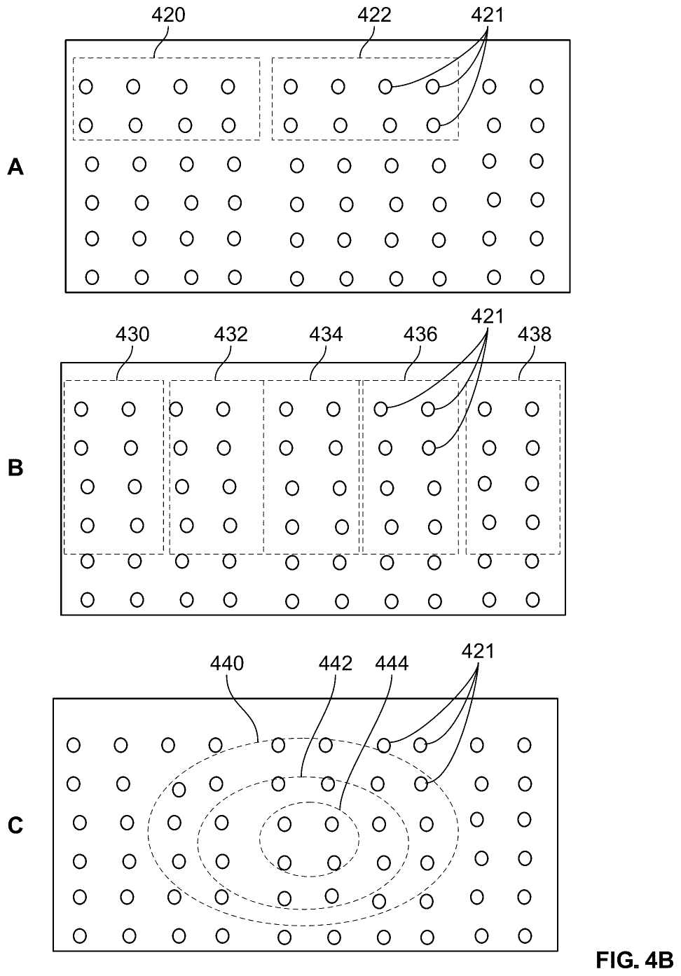

FIG. 3 shows an example of interferential stimulation. In some embodiments IVS system 100 may use transcranial magnetic stimulation (TMS) and transcranial direct current stimulation (TDCS). IVS system 100 may in some embodiments also use off-the-shelf modeling tools such as Soterix HD-Explore to simulate the conductivity of different types of tissue such as grey/white matter, cerebrospinal fluid, skull, and skin. Combining brain modeling with temporal interference modeling may enable accurate targeting of stimulation location and intensity as shown in FIG. 4A.

FIG. 3 shows a diagram of a human head 300. Electrode pairs 302 and 304 are placed near the vestibular system and in an embodiment emit waves of electrical currents 306 and 308 of frequencies 2.01 kHz and 2 kHz, respectively. The currents may be alternating current as alternating is more precise than direct current in the context of interferential stimulation. These frequencies 2.01 kHz and 2 kHz are example frequencies. An embodiment may use frequencies ranging from 1-200 Hz, which roughly maps onto the range of natural neuronal firing frequencies. Interferential stimulation parameters would in some embodiments include hard limits on frequency and charge density to prevent unwanted stimulation. For example, a hard minimum of 1 Khz frequency safely prevents the individual stimulation from each electrode from stimulating unintentional tissue. IVS system 100 may use two separate sets of limits: one for the individual electrodes and one for the predicted created interference pattern. In an embodiment the limit on the charge density of a stimulation pulse is 30 μC cm−2 for electrodes having a geometric surface area of 0.06 cm2. In an embodiment default values can be adjusted based on electrode size and stimulation parameters. Baseline stimulation parameters in some embodiments could target regions centered in the predicted locations of the left and right vestibular systems in the average human brain. For example, baseline stimulation parameters might stimulate 2×1″ radius spheres positioned 3″ inside the ear on a line connecting the user's ears. The size and location of the targeted region could be modified by the size of the headset being worn or a user calibration profile. A user calibration profile may also include a heat map of skin conductivity around their scalp, to assist with calculating parameters. It may also include curves for conductivity over the course of a session or as a function of exertion (i.e., sweat).

As seen in the figure, the waves of electrical currents 306 and 308 travel through head 300. When waves 306 and 308 intersect, they create within head 300 envelop 310 which is an electrical field with an interference pattern that causes interferential stimulation. As discussed above, the interferential stimulation, here represented as envelop 310, can reach areas at specified distances from the electrode 302 and 304 and therefore target specific sections, by distance, of the head 300. The resulting electrical field of the interferential stimulation has a bias in one direction. Such electrical fields are known to create the perception of acceleration in a person's vestibular system 201 in the same direction of the bias. In an embodiment electrode pairs 302 and 304 are electrode pairs that each include a source and a sink. Both electrodes, that is one pair of source and sink electrodes, are placed near the head 300 to transmit an electrical current (e.g., alternating current) in an embodiment. To transmit multiple currents and cause the interference pattern which can target specifical points, multiple pairs of electrodes may be placed near the head 300.

Increasing the number of electrodes, and thereby increasing the number of currents traveling through head 300, can increase precision regarding the desire to target specific areas or distances. FIG. 4A illustrates how an interference stimulation pattern becomes more focused as the number of electrode pairs increases as shown through a laboratory simulation. Pattern mapping (a) shows targeted region 401. Targeted region 401 is the result of one pair of electrodes emitting a current through tissue. Targeted region 401 is relatively long. By comparison pattern mapping (b) shows targeted region 402 which is the area targeted by three electrode pairs on the same tissue as pattern mapping (a). Compared to 401, region 402 is smaller. Because the targeted region 402 is smaller than 401, targeted region 402 is more precise in targeting than region 401. Similarly, pattern mapping (c) shows targeted region 403. Targeted region 403 is the result of six pairs of electrodes emitting currents through tissue. Compared to 401 and 402, targeted region 403 is the smallest and most round. Because the targeted region 403 is smaller than 401 and 402, targeted region 402 is more precise in targeting than regions 401 and 402. An embodiment may use sixteen pairs of electrodes. In another embodiment, thirty-six pairs conduct interferential waves. An embodiment may use hundreds or thousands of electrode pairs. The dramatic improvement between the regions 401, 402, and 403 illustrates how precise interferential stimulation in such embodiments may be.

Such an embodiment is shown in FIG. 4B. That FIG. 4B shows different example configurations of electrodes which may be modified to produce various perceived accelerations. The number of electrodes may vary depending on preferences, application, or otherwise as well. IVS system 100 may test different configurations over the course of a calibration cycle, for example while updating a calibration profile as discussed below as element 707. During calibration the IVS system 100 begins with one configuration, for example configuration A. In configuration A group 420 of electrodes 421 emits a current of 2.0 khz while group 422 emits a current of 2.1 kHz. The currents create an interference pattern resulting in vestibular stimulation. The effects of the vestibular stimulation may be detected through a reactionary head movement (that is, a person's subjective perception of acceleration, resulting from the vestibular stimulation, may be inferred by observing a reactionary head movement). The vestibular system 201 senses acceleration and via the efferent nerve system sends that signal reporting its perception to the relevant brain regions. The interferential stimulation impacts the perception of acceleration by intercepting this process. The interferential stimulation may cause the efferent nerve system to send signals reflecting, in place of information the vestibular system 201 detects, the acceleration information the interference pattern delivered. Then, certain types of interferential patterns result in certain movements, which for example IMU 502 may detect and measure. If the measured effects are not as desired and indicate that a user does not experience the intended acceleration, the parameters of the currents may be updated in a second attempt to deliver the correct perceived acceleration. In one example, updating the currents includes dividing the electrodes into further subsections than what is seen in FIG. 4B, where each electrode in a group delivers a current with the same parameters. The additional subsections may be along a linear axis for example as shown in configuration group B with groups 430-438 of electrodes 421. Alternatively, the additional subsections may follow a pattern such as a circular pattern as seen in configure C with groups of electrodes 440-444. The subsections may be placed according to other criteria such as predicted size of a vestibular system or a calculated vector representing acceleration. In an embodiment, some electrodes may be instructed to omit no current. Configuration A shows 32 electrodes is a plane of 8 and 4. Another embodiment incudes electrodes in an arrangement of 50 by 20 electrodes. The plane of 50 by 20 electrodes may be reduced to 30 by 20 if the IVS system 100 so directs for example. In additional to varying configurations, the output of each current may be changed as well. In an embodiment the output of electrodes is updated incrementally. For example, if the scenario of group of electrodes 420 emitting a current of 2.0 khz while group 422 emits a current of 2.1 kHz is found to be to be ineffective, group 422 may next omit a current of 2.11 kHz, and then 2.12 kHz and so on until satisfactory parameters are reached.

In an embodiment, a calibration cycle begins with configuration A with group 420 at 2 kHz and group 422 at 2.1 kHz. In an embodiment the initial configuration and currents are based on a calibration profile. In an embodiment the calibration profile includes a list of electrode configurations documented to induce known results and the initial configuration and current parameters are chosen from the list. For example, in an embodiment, a perception of 10 m/s² may typically result in head movement at 0.5 cm/s². After the IVS system 100 delivers the example currents and configuration, it detects that the resulting interference results in a passenger's head moving forward. However, the expected movement, calculated for example in accordance with that passenger's calibration profile, of the passenger's head was to remain still (e.g., because the calibration profile has recorded that the resulting interference either cancels perceived forward movement or induces equal and opposite movement). The IVS system detects the undesired movement and adjusts the parameters, such as for example, the amplitude, frequency, and phase of the interferential electrical waves. This may in an embodiment adjust current density (intensity of perceived acceleration) and bias of the created electrical field (direction of perceived acceleration). For example, the IVS system 100 adjusts configuration A so that group 422 delivers waves of 2.2 kHz. Next it tries 2.3 kHz for example. At one point it may reduce group 420 to 1.9 kHz as well. The calibration continues in this or suitable similar manner (e.g., for the entire duration of a vehicle ride or XR presentation). When the desired resulting movement occurs, the IVS system makes no further adjustments. Other arrangements may be appropriate for other acceleration directions as well. In some embodiment, when the desired resulting movement occurs, IVS system 100 updates a user calibration profile to include the successful data point.

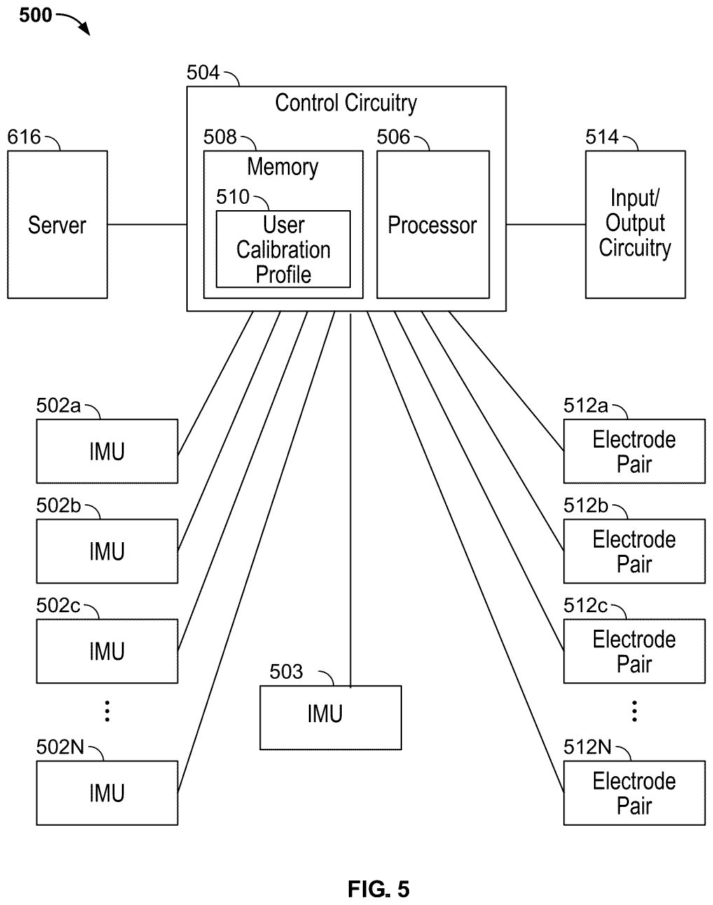

FIG. 5 shows an example architecture 500 of an IVS system, such as the IVS system 100, that is configured to use interferential stimulation to target symptoms of motion sickness. The IVS system 100 includes at least one IMU 502. The IMU detects acceleration and in some embodiments is connected to a head of an individual. In an embodiment the IMU 502 is connected via a wearable device such as a headset. The IVS system 100, including a wearable device, may include multiple IMUs 502. FIG. 5 shows multiple IMUs 502: IMU 502a, IMU 502b, IMU 502c, through IMU 502N. The IMUs 502 collect data and communicate the data to the control circuitry 504 where the data is processed in processor 506. An embodiment may include an IMU 503 in a vehicle such as vehicle 150 where IMU 503 monitors the acceleration of the vehicle. Processing includes for example determining if collected acceleration data received from IMU 502 matches predicted acceleration data for example. The control circuitry 504 also includes memory 508. Memory 508 stores data. In an embodiment the memory 508 includes data containing a user calibration profile 510. The IVS system 100 also includes high density electrode arrays made up of electrode pairs 512 which may be part of a headset or wearable device as well. FIG. 5 shows multiple electrode pairs 512, electrode pair 512a, electrode pair 512b, electrode pair 512c, through electrode pair 512N. In an embodiment the IVS system 100 includes additional sensors such as a sensor that detects sweat levels on the skin's surface or a heart rate monitor. IVS system 100 in some embodiments also includes input/output circuitry 514 for receiving and sending data such as acceleration data and user preferences. In an embodiment IVS system 100 is further connected to a server 516 for cloud processing and may receive further data via the server. In such an embodiment the server 516 connects to IVS system 100 through a network via a communication link.

In an embodiment, a closed-loop interferential stimulator targets the vestibular system 201 of a passenger in an automobile 150 or another individual otherwise perceiving acceleration. An embodiment enacts the closed-loop interferential stimulator using a wearable device 600 seen in FIG. 6. Wearable device 600 for example includes high-density electrode pair 512, such as electrode pair 512a, connected to the wearable device 600 in a position and manner to be worn around each ear of a wearer. In an embodiment electrode pair 512 is a dry electrode. In an embodiment an electrode pair 512 is embedded in a ring around an ear as with over the ear headphones. The wearer may be a passenger in a moving vehicle for example. Wearable device 600 also includes in an embodiment one or more embedded IMUs 502, such as IMU 502a, that detect acceleration. These IMUs 502 detect for example acceleration of the head of the wearer. Wearable device 600 also may include a band 603 or other mechanism for attaching the elements to the head. Wearable device 600 may also include processing circuitry 606 and a communication interface in some embodiments. In some embodiments wearable device 600 includes additional sensors such as those that detect sweat level, heart rate, temperature, or other qualities. Additional sensors may provide additional data. For example, a thermometer might collect forehead temperature of a person wearing wearable device 600, where the forehead temperature is directly related to a predicted sweat level which might impact skin electrical conductivity.

The electrode pair 512 in an embodiment has the potential to stimulate the sensations of angular and linear acceleration across all three axes shown in FIG. 2B. A person may move his or her head in response to perceiving stimulation. The IVS system 100 in an embodiment calculates parameters of interferential stimulation to stimulate the sensations of angular and linear acceleration in the desired directions. The parameters may in an embodiment include frequency, wavelength, and amplitude of waves generated with one or more electrode pairs 512. The waves then stimulate the vestibular system 201 of the wearer of wearable device 600 in accordance with interferential stimulation as seen in FIGS. 3 and 4. Vestibular stimulation may cause the head of the wearer to move in the direction perceived as a reaction to the stimulation. The movement of the head in an embodiment enables the proposed IVS system 100 to validate and update stimulation parameters in real time using head motion data from a variety of potential sources including IMUs 502. In an embodiment stimulating motion in the opposite direction prevents motion sickness for the wearer of wearable device 600.

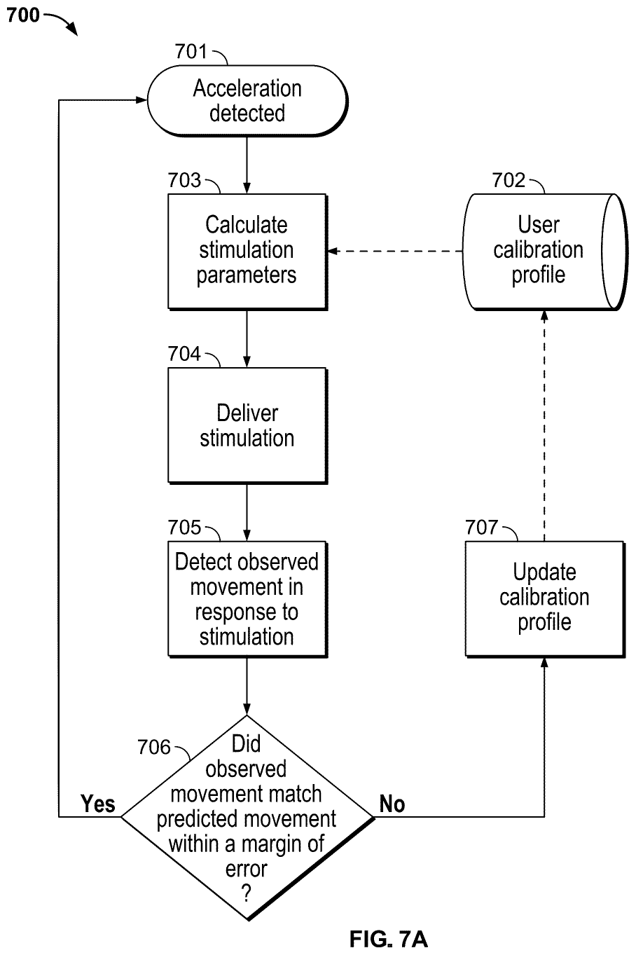

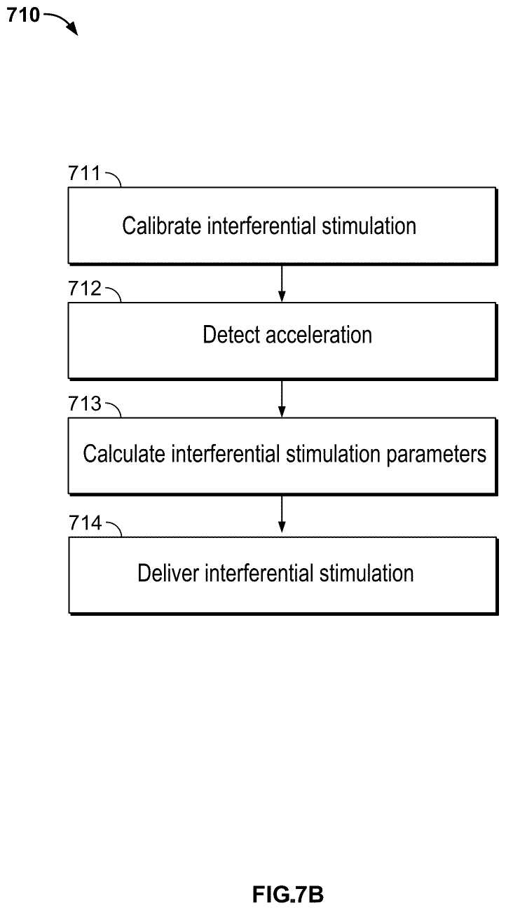

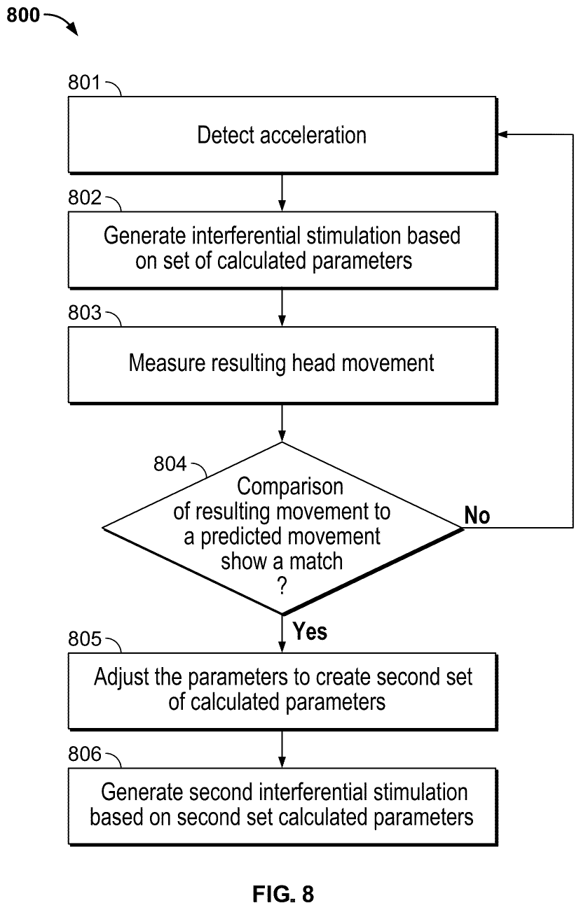

A process 700 of an embodiment of the present disclosure is shown in FIG. 7A. The process 700 may be implemented, in whole or in part, by control circuitry 504 of IVS system 100, including processor 506. For example, one or more of the aforementioned systems or devices, such as those shown in FIG. 5, may execute one or more instructions or routines stored to memory or storage of a device to implement, in whole or in part, the process 700.

In an embodiment the IVS system 100 detects acceleration at step 701. This acceleration may be the result of, for example, a moving vehicle in which a passenger is traveling. An accelerometer such as an IMU within the vehicle, for example, may in some embodiments detect the acceleration in step 701. IVS system 100, using, for example processor 506, receives such information from, for example, an IMU 502 connected to a control circuitry 504. In an embodiment the acceleration is virtual acceleration reproduced on a screen, such as in a video game, XR interface, or in a movie. In such an embodiment, the IVS system 100, using for example processor 506, receives information of acceleration directly from the displayed content, the video game or movie feed or metadata for example. Indirect measures such as simultaneous localization and mapping (S.L.A.M.) based on visual features from a video feed may also detect acceleration. A link transferring acceleration data may be for example a wireless or wired link.

At step 702 the IVS system 100 receives a user calibration profile. In an embodiment the user calibration profile is collected before process 700 begins. In an embodiment the process 700 includes a start-up period or other calibration period in which it gathers calibration data. In an embodiment process 700 functions independent of calibration and instead relies on a provided model. Using the received acceleration information IVS system 100, using for example processor 506, then at step 703 calculates stimulation parameters for guiding the electrode outputs. Stimulation parameters may be for example wavelength, amplitude, and frequency of a wave produced by electrical currents. In an embodiment, the processor 506 or other circuitry calculates the parameters. In an embodiment the step 703 also incorporates a user calibration profile, which identifies patterns of stimulation that correspond to different axes of linear and angular acceleration. The processor 506 may calculate parameters that are likely to create the perception of the desired acceleration, the desired acceleration being the acceleration detected by a person's vision, whether that be a person in a moving vehicle or a person watching a screen. In an embodiment the processor 506 calculates a baseline or desired perceived acceleration. For example, in an embodiment it calculates parameters to deliver interferential stimulation that is known to create the perception of no acceleration. IVS system 100 then for example informs the appropriate electrode outputs to create a wave or waves with the specified parameters.

At step 704 in an embodiment the system 100 delivers the stimulation in accordance with the parameters the IVS processor 506 calculated at step 703. The stimulation is for example electrode output in the form of one or more electrical currents. The electrodes in an embodiment are attached to a head of a passenger in such a manner that the stimulation that the IVS system 100 delivers reaches a targeted area of the brain of the passenger. One targeted area of the head of the passenger is for example the vestibular system of the brain. The wave or waves then counteracts or override the perceived acceleration. In an embodiment, the stimulation that the IVS system 100 delivers causes acceleration of the head of the passenger in reaction to the perceived acceleration. At step 705 IVS system 100 detects observed acceleration in response to the stimulation. In an embodiment the IVS system 100 detects this acceleration through an accelerometer such as one or more IMUs 502 attached to the head of the passenger via a headset or XR headset for example. In an embodiment the IVS system 100 detects reactionary head movement of the passenger. The accelerometer may send information regarding the acceleration or acceleration of the head to the IVS system 100 through for example a wired link. For example, the IMU 502 may collect data regarding acceleration and send that information to the control circuitry 504. In an embodiment, head acceleration in response to stimulation is detected via external sensors such as cameras, time-of-flight, or Lidar.

At step 706 IVS system 100 determines whether or not the observed acceleration matches a predicted acceleration. The predicted acceleration in some embodiments represents an acceleration at which the passenger feels reduced motion sickness. In an embodiment this determination is performed using processing circuitry 506. In an embodiment the determination is made within a given or calculated margin of error. In an embodiment if the system 100 determines at step 706 that the observed acceleration matches a predicted acceleration, IVS system 100 ends the process 700 for that acceleration and returns to step 701 where it receives the next detected acceleration. In an embodiment if the IVS system 100 determines at step 706 that the observed acceleration does not match a predicted acceleration, the IVS system 100 may update a calibration profile at step 707. In this scenario, the IVS system may run a calibration cycle similar to that disclosed in FIG. 4B. IVS system 100 in some embodiments may incorporate the updated user calibration profile into the existing user calibration profile and use this updated user profile again returning to step 702.

This process 700 may be carried out in the example of a passenger reading in a moving automobile in an embodiment, similar to the IVS system 100 and example shown in FIG. 1A. In one such embodiment, the automobile is moving forward, creating constant acceleration in the forward direction as the automobile speeds up and slows down during the course of the drive. For example, the automobile will experience deceleration when it stops at a red light and acceleration when the light turns green. Similarly, it will decelerate when an automobile in front of it slows down and will accelerate when it reaches an open road. Initially the passenger reading may feel motion sick. The passenger in this embodiment is not looking out the window, but instead looking at still words on a page of a book. The vestibular system 201 of the passenger however detects acceleration as the automobile drives since the passenger is moving with the automobile. The discrepancy between what the passenger's vision detects and what the passenger's vestibular system detects causes the passenger to feel motion sick.

In such an embodiment the passenger may wear the wearable device 600 containing accelerometers or IMUs 502 and electrode pairs 512. In this scenario and embodiment, when the automobile slows down at a red light, the IVS system 100 detects deceleration (e.g., acceleration, in a direction of the longitudinal axis of the automobile, opposite the direction of movement of the automobile). Without other stimulation, the passenger's vestibular system may sense this deceleration while the passenger's eyes will not, potentially causing the passenger to feel motion sick. The IVS system 100 then may calculate stimulation parameters, or parameters of interferential stimulation, which are known to cause the vestibular system to sense a specific acceleration, in this case no acceleration to match what the reading passenger's eyes are sensing. The stimulation parameters calculation may incorporate a range of information, for example, the calculated stimulation may also include calibration data conveying for example, a location of a specific passenger's vestibular system or the passenger's individual reception to interferential stimulation. The stimulation parameters may include wavelength, amplitude, and frequency of an electrical current output by electrodes. In the embodiment of the reading passenger in a moving vehicle, once the IVS system 100 has calculated the parameters, the IVS system 100 delivers the stimulation in accordance with the parameters to the passenger through the electrodes in the wearable device 600. The electrodes may be positioned around the ear of the passenger and the electrical currents the electrodes deliver may reach an area or areas of the passenger's brain that cause the passenger to experience acceleration.

In the embodiment the passenger's head moves through space as the vehicle moves. An IMU 502 detects the acceleration related to this motion. The delivered interferential stimulation may cause additional head acceleration in reaction to the stimulation and the IN/U 502 may detect this acceleration as well. The IVS system 100 detects the acceleration of the passenger's head and determines that it matches a predicted acceleration. For example, if the interferential stimulation should create feelings of no movement in the vestibular system, as is the case in the embodiment of a reading passenger, the expected movement of the head is no reactionary movement. An embodiment may further include at least one IMU 503 devoted to detecting acceleration of the vehicle 150 and the predicted acceleration of the head may account for the detected acceleration of the automobile. For example, in an embodiment, the detected acceleration of a head may be the delta or difference between acceleration of the head expected from experienced acceleration from the vehicle 150 and the actual measured acceleration of the head IMU 502 collects. In an embodiment, IVS system 100 calibrates before the vehicle 150 begins accelerating such that IVS system 100 determines a predicted acceleration of the head without the additional variable that the automobile acceleration introduces. In the embodiment of an XR headset (e.g., a VR headset), the expected movement is movement in line with the acceleration the eyes detect. This determination indicates to the IVS system 100 that the interferential stimulation has been successful. The IVS system 100 then repeats this process when a next new acceleration is detected. For example, when the automobile accelerates upon the light turning green. Again, in the scenario of the passenger reading in an automobile, the IVS system 100 may calculate and deliver through electrodes interferential stimulation that is intended to cause the passenger to feel still, the same acceleration the passenger's eyes detect, although the body within the automobile is accelerating. The IVS system 100 delivers again a calculated stimulation using a first calculated set of parameters. The passenger's head moves forward in response, indicating that the passenger likely felt forward acceleration. The IMUs detect this motion and report it to the IVS system 100. The IVS system 100 determines that the acceleration of the head was not the expected response. As a result, the IVS system 100 updates the parameters and delivers a second stimulation. The IVS system 100 again detects a response to the second stimulation. This time, the response is as expected and the IVS system 100 moves on to detect a next acceleration. In an embodiment, the responsive motions of the head may be minimal and/or detected over the course of milliseconds, allowing the IVS system 100 the opportunity to correct the stimulation before the passenger feels its effects.

The IVS system 100 may also take place in the context of virtual reality or similar visual simulation. The IVS system 100 may directly receive virtual acceleration data from the visual stimulation source to inform the IVS system 100 of the acceleration the viewer's eyes perceive in such embodiments. In an embodiment acceleration is detected based on user input such as a player in a video game moving a joystick to make an avatar run faster. In one example embodiment a player watches a video of a video game involving high speed vehicles. The screen may create an immersive feel for the player. The player then in an embodiment may experience a sensation of acceleration based on what he or she sees based on the viewpoint on the screen. However, the vestibular system of the player senses no acceleration. The player's brain may receive conflicting information, and the player may feel motion sick. In such an embodiment IVS system 100 may detect acceleration on the screen and calculate electrode output by way of output parameters that would likely cause the player to perceive the same acceleration through the vestibular system 201. The electrode output may create interferential stimulation fields and the IVS system's 100 calculations may include parameters including wavelength, frequency, and amplitude figures that best account for the detected acceleration. In an embodiment where the player is wearing wearable device 600, the IVS system 100 may next deliver the electrode output, and by extension, stimulation, through the electrode pairs 512. The resulting fields may reach the vestibular system of the player, causing the player to experience the perception of acceleration. In response, the player may instinctively move his or her head in the direction of the perceived acceleration. The IMUs 502 or other accelerometers on wearable device 600 may detect the motion of the head. The IVS system 100 may receive information regarding the motion of the head from IMUs 502 and determine whether the interferential stimulation caused a movement that matches a predicted motion of the player's head in response to the interferential stimulation. If the movement of the head matches the predicted movement, the IVS system 100 may end its process. If the movement does not match however the IVS system 100 may adjust outputs from the electrodes and again detect and analyze the player's head movement.

In an embodiment, the IVS system 100 completes a dedicated calibration phase in which the IVS system 100 cycles through patterns of stimulation to identify which patterns of stimulation correspond to perception of different axes and intensities of linear and angular acceleration. Users may require different parameters for example due to factors such as skull density, resistance, sweat patterns, and size. Calibration may account for differences between users to improve performance of IVS system 100 for each user. In an embodiment the calibration phase is automatic. In an embodiment the calibration phase occurs at the beginning of a use case of the present IVS system 100. For example, when a passenger begins a ride in a moving vehicle wearing a wearable device 600 with the disclosed IVS system 100, the IVS system 100 may begin calibration without input from the user. In this scenario the IVS system 100 may record resulting head movements after transmitting various generated patterns of interferential stimulation. It may compare the resulting movements to predicted movements. After a series of trials of generating waves and comparing their resulting movement to a predicted movement, the IVS system 100 is able to determine the correct parameters corresponding to intended perceived accelerations, by way of resulting head movement, and likely to cause the intended perceptions. The IVS system 100 may save these parameters in a user calibration profile. In an embodiment a user calibration profile includes a lookup table of sample or past accelerations and directions and their corresponding parameters for a given user. In an embodiment the user calibration profile contains data such as values representing skull density, head size, brain region locations, resistance, and sweat habits. In an embodiment the IVS system 100 begins with a model which includes an estimated value for various variables such as for example vestibular system location in space and electrical resistance of the skull. In an embodiment the IVS system 100 calculates the set of parameters using data from the model. In an embodiment after delivering interferential stimulation using parameters based on the model, the IVS system 100 measures the accuracy of the delivered interferential stimulation using a reactionary movement of a person's head. The IVS system 100 in an embodiment may then adapt the parameters of the interferential stimulation based on the determined accuracy of the delivered interferential stimulation.

In an embodiment, the IVS system 100 does not have a calibration profile. In an embodiment the IVS system 100 gradually increases stimulation strength as it refines a default or existing calibration profile over the course of an experience. In an embodiment the IVS system 100 calibrates during a specified calibration session. For example, the IVS system 100 may calibrate the IVS system 100 in response to a selection to “calibrate.” The selection to calibrate may occur at a time when the headset 600 is not otherwise in use. In an embodiment calibration begins by identifying the user's motor threshold (MT)—the minimum stimulation intensity required to elicit noticeable head movement in a single direction. Perceived acceleration can be inferred based on head acceleration that is measured via accelerometers such as IMU 502 mounted in a headset 600, glasses, a force platform/pressure plate, or with electromyographic (EMG) sensors placed on relevant muscles. Calibration may involve modeling stimulation parameters that generate interference patterns of 0.1 Hz with a direction facing behind the user. In an embodiment the IVS system 100 gradually increases the resulting stimulation frequency at a rate of 0.1 Hz/s2 while maintaining a charge density below the limit of 30 μC cm−2 at the electrode sites and the interference pattern. Once the motor threshold has been identified, the angle of movement may be compared to the predicted simulated acceleration angle. If the angular difference is greater than a threshold (e.g., 5°) then stimulation parameters may be adjusted until the desired angle of simulated acceleration is achieved. Such a process could be completed step-wise (e.g., in 100 increments) separately for each rotational and linear axis, to identify which individual axes of acceleration can be effectively simulated. Once rotational and linear axes have been identified in isolation, they could be sequentially combined in a similarly step-wise fashion to identify which combinations of axes can be affected.

In an embodiment the wearable device 600 or the IVS system 100 includes an electromyography (EMG) sensor. The EMG sensors may be embedded in wearable device 600 or worn as a separate sensor for example. In an embodiment one or more EMG sensors detect activation of muscles, for example head and neck muscles, before they contract. In an embodiment the IVS system 100 uses feedback from EMG sensors during calibration to determine whether or not a particular stimulation has the desired effect. For example, the IVS system 100 may deliver interferential stimulation and, using one or more EMG sensors, detect activation or movement of neck or head muscles. The activation or movement of the muscles indicates head movement indicative of a perception of acceleration. The indication of perceived acceleration may signify that particular parameters have delivered the intended effect or not.

A process 710 of an embodiment of the present disclosure in which calibration occurs before detected acceleration is shown in FIG. 7B. The process 710 may be implemented, in whole or in part, by control circuitry 504 of IVS system 100, including processor 506. For example, one or more of the aforementioned systems or devices, such as those shown in FIG. 5, may execute one or more instructions or routines stored to memory or storage of a device to implement, in whole or in part, the process 710. At step 711 of process 710, the IVS system 100 calibrates interferential stimulation parameters using for example processor 506. In an embodiment this calibration occurs through a trial and error process discussed above in which one ore more IMUs such as IMU 502 detects and reports head movement resulting from various interferential stimulation patterns the electrodes pairs 512 deliver. The IVS system 100 records results of calibration in an embodiment in a memory such as memory 508 containing user calibration profile 510. When calibration is complete, the IVS system 100 may detect acceleration at step 712 and at step 713 calculate, based on a combination of the user calibration profile 510 and the detected acceleration, parameters for interferential stimulation. At step 714 IVS system 100 may deliver interferential stimulation at the calculated parameters. In an embodiment the IVS system 100 then returns to step 712 to detect a second acceleration.

FIG. 8 illustrates an example process 800 of an embodiment of the disclosure. The process 800 may be implemented, in whole or in part, by control circuitry 504 of IVS system 100, including processor 506. For example, one or more of the aforementioned systems or devices, such as those shown in FIG. 5, may execute one or more instructions or routines stored to memory or storage of a device to implement, in whole or in part, the process 800. At step 801 control circuitry (e.g., control circuitry 504 of FIG. 5) detects an acceleration as an acceleration data stream. This acceleration may be through data received by for example IMUs 502a through 502N in an embodiment that is correcting for detected acceleration in a moving vehicle. In an embodiment that is corrected for perceived acceleration seen on a screen, such as XR headset or IMAX theatre, the control circuitry may receive acceleration data directly from content. In response to the detection, at step 802 the IVS system 100, through for example control circuitry 504, generates using multiple electrodes connected to a human head, such as electrode pairs 512a through 512N, an interferential stimulation pattern causing, as a reaction, a first movement of the human head, wherein the interferential stimulation pattern is based on a calculated set of parameters. In some embodiments the interferential stimulation causes the vestibular system to detect no acceleration, such as in the case of a passenger in a moving vehicle. In some embodiments, the interferential stimulation causes the vestibular system to detect acceleration mirror that on a screen, such as in the case of an individual wearing a XR headset. In an embodiment the IVS system 100 performs step 802 without prompt. In an embodiment the processor 506 calculates the set of parameters. The calculation may be based on for example data collected from for example IMU 502 or other sensors or feed in communication with processor 506. The IVS system 100 next at step 803 measures a resulting movement of the human head, where the resulting movement of the human head occurred in response to the interferential stimulation pattern. In an embodiment the IVS system 100 measures this movement using data from one or more IMUs 502.

At step 804 the IVS system 100 compares the resulting movement to a predicted movement, wherein the predicted movement is based on a calibration profile and an intended perceived acceleration. For example, if the interferential stimulation should create feelings of no movement in the vestibular system, as is the case in the embodiment of a reading passenger, the expected movement of the head is no movement. In the embodiment of a XR headset, the expected movement is movement in line with the perceived acceleration. The IVS system 100 may compare the movements using processor 506. The example, IVS system 100 obtains the calibration profile as described above. If the resulting movement matches the predicted movement, or answers “yes” at step 804, the process 800 returns to step 801. If the resulting movement does not match the predicted movement, or answers “no” at step 804, at step 805 the IVS system 100 adjusts the set of parameters based on the comparing to create an adjusted set of parameters. Next the process 800 moves to step 806 where the IVS system 100 in an embodiment generates using the multiple electrode pair connected to the human head, such as electrode pairs 512, a second interferential stimulation pattern based on the adjusted set of parameters, causing a second movement of the human head. The IVS system 100 may then repeat steps 801 through 806 until a desired head movement is detected.

The processes described above are intended to be illustrative and not limiting. One skilled in the art would appreciate that the steps of the processes discussed herein may be omitted, modified, combined, and/or rearranged, and any additional steps may be performed without departing from the scope of the disclosure. More generally, the above disclosure is meant to be exemplary and not limiting. Only the claims that follow are meant to set bounds as to what the present disclosure includes. Furthermore, it should be noted that the features and limitations described in any an embodiment may be applied to any other embodiment herein, and flowcharts or examples relating to an embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

1. A method comprising:
receiving an acceleration data stream;
generating using multiple electrodes connected to a human head, an interferential stimulation pattern causing a first movement of the human head, wherein the interferential stimulation pattern is based on the acceleration data stream and a set of parameters;
measuring a resulting movement of the human head, wherein the resulting movement of the human head occurred in response to the interferential stimulation pattern;
comparing the resulting movement to a predicted movement, wherein the predicted movement is based on a calibration profile;
adjusting the set of parameters based on the comparing to create an adjusted set of parameters; and
generating using the multiple electrodes connected to the human head, a second interferential stimulation pattern based on the adjusted set of parameters.

2. A method as claimed in claim 1 wherein the following are repeated until results of comparing the resulting movement to the predicted movement do not improve:
comparing the resulting movement to the predicted movement;
adjusting the set of parameters based on the comparing to create the adjusted set of parameters; and
generating, using the multiple electrodes, the second interferential stimulation pattern based on the adjusted set of parameters.

3. A method as claimed in claim 1 wherein the following are repeated until a result of comparing the resulting movement to the predicted movement falls within a threshold:
comparing the resulting movement to the predicted movement;
adjusting the set of parameters based on the comparing to create the adjusted set of parameters; and
generating, using the multiple electrodes, the second interferential stimulation pattern based on the adjusted set of parameters.

4. A method as claimed in claim 1 wherein the multiple electrodes are positioned around the ears of the human head.

5. A method as claimed in claim 1 wherein the multiple electrodes are positioned in a headset.

6. A method as claimed in claim 1 wherein the set of parameters is calculated based on matching the acceleration data stream with the movement of the human head.

7. A method as claimed in claim 1 wherein the set of parameters is calculated based on counteracting the acceleration data stream.

8. A method as claimed in claim 1 wherein the acceleration data stream is received from at least one Inertial Measurement Unit (IMU) measuring acceleration of a vehicle.

9. A method as claimed in claim 1 wherein the acceleration data stream is received directly from an extended reality (XR) simulation.

10. A method as claimed in claim 1 wherein the set of parameters include a frequency parameter, a wavelength parameter, and an amplitude parameter of the interferential stimulation pattern.

11. A system comprising:
processing circuitry configured to:
receive an acceleration data stream;
generate using multiple electrodes connected to a human head, an interferential stimulation pattern causing a first movement of the human head, wherein the interferential stimulation pattern is based on the acceleration data stream and a set of parameters;
measure a resulting movement of the human head, wherein the resulting movement of the human head occurred in response to the interferential stimulation pattern;
compare the resulting movement to a predicted movement, wherein the predicted movement is based on a calibration profile;
adjust the set of parameters based on the comparing to create an adjusted set of parameters; and
generate using the multiple electrodes connected to the human head, a second interferential stimulation pattern based on the adjusted set of parameters.

12. A system as claimed in claim 11 wherein the following are repeated until results of comparing the resulting movement to the predicted movement do not improve:
comparing the resulting movement to the predicted movement;
adjusting the set of parameters based on the comparing to create the adjusted set of parameters; and
generating, using the multiple electrodes, the second interferential stimulation pattern based on the adjusted set of parameters.

13. A system as claimed in claim 11 the processing circuitry further configured to repeat the following until a result of comparing the resulting movement to the predicted movement falls within a threshold:
compare the resulting movement to the predicted movement;
adjust the set of parameters based on the comparing to create the adjusted set of parameters; and
generate, using the multiple electrodes, the second interferential stimulation pattern based on the adjusted set of parameters.

14. A system as claimed in claim 11 wherein the multiple electrodes are positioned around the ears of the human head.

15. A system as claimed in claim 11 wherein the multiple electrodes are positioned in a headset.

16. A system as claimed in claim 11 wherein the set of parameters is calculated based on matching the acceleration data stream with the movement of the human head.

17. A system as claimed in claim 11 wherein the set of parameters is calculated based on counteracting the acceleration data stream.

18. A system as claimed in claim 11 wherein the acceleration data stream is received from at least one Inertial Measurement Unit (IMU) measuring acceleration of a vehicle.

19. A system as claimed in claim 11 wherein the acceleration data stream is received directly from an extended reality (XR) simulation.

20. A system as claimed in claim 11 wherein the set of parameters include a frequency parameter, a wavelength parameter, and an amplitude parameter of the interferential stimulation pattern.

21-50. (canceled)
